(19) United States
(12) Patent Application Publication
(10) Pub. No.: US 2021/0169344 A1
(43) Pub. Date: Jun. 10, 2021

(54) BLOOD PRESSURE MEASURING MODULE

(71) Applicant: Microjet Technology Co., Ltd., Hsinchu (TW)

(72) Inventors: Hao-Jan Mou, Hsinchu (TW); Yung-Lung Han, Hsinchu (TW); Chi-Feng Huang, Hsinchu (TW); Chang-Yen Tsai, Hsinchu (TW); Wei-Ming Lee, Hsinchu (TW); Chun-Yi Kuo, Hsinchu (TW); Yang Ku, Hsinchu (TW)

(73) Assignee: Microjet Technology Co., Ltd., Hsinchu (TW)

(21) Appl. No.: 17/081,543

(22) Filed: Oct. 27, 2020

(30) Foreign Application Priority Data
Dec. 6, 2019 (TW) 108144818

(51) Int. Cl. A61B 5/0205; A61B 5/00; A61B 5/046; A61B 5/1455

(52) U.S. Cl. CPC A61B 5/0205; A61B 5/681; A61B 5/002; A61B 5/0022; A61B 5/7264; A61B 5/02405; A61B 5/046; A61B 5/14551; A61B 2562/0247; A61B 2562/0219; A61B 5/746

(57) ABSTRACT

A blood pressure measuring module is provided and includes at least one module body, at least one gas transportation device, at least one sensor. The module body is connected to an airbag, so as to control inflation and deflation operation of the airbag. The gas transportation device controls gas to flow. The sensor measures the variety of the gas pressure in the airbag or the pressure in contact with the user's skin. Gas transportation is formed as the gas transportation device is driven. The gas is guided into the module body and then is concentrated in the airbag, so that the airbag is inflated to perform the blood pressure measurement. The sensor measures the gas pressure accumulated in the airbag or the pressure in contact with the user's skin, and the user's blood pressure information is calculated.

### FIELD OF THE INVENTION

The present disclosure relates to a blood pressure measuring module, and more particularly to a blood pressure measuring module applied to a wearable blood pressure measuring device.

### BACKGROUND OF THE INVENTION

Currently, in all fields, the products used in many sectors such as pharmaceutical industries, computer techniques, printing industries or energy industries are developed toward elaboration and miniaturization. Among them, a blood pressure measuring module is regarded as a key technology. Therefore, how to create an innovative structure to break through the technical bottleneck is an important content of development. For example, in the pharmaceutical industries, many instruments or equipment, such as blood pressure measuring devices, which needs to be actuated by a driving force of fluid. Usually, a conventional motor and a gas valves are utilized to achieve the purpose of gas transportation. However, since the volumes of the conventional motor and the gas valve are limited, it is difficult to reduce the entire volume of such equipment. Namely, it is difficult to achieve the goal of minimization. Moreover, it is impossible to make it portable. On the other hand, when the conventional motor and the gas valve are actuated, noise is generated, and it results in inconvenience and uncomfortable use.

Therefore, a blood pressure measuring module applied to a wearable blood pressure measuring device is provided for the use of the industry, so as to overcome the above-mentioned drawbacks in the prior art and make the conventional instrument or equipment having a gas transportation device to meet the goals of small size, miniaturization and quietness, and having an ability of rapidly transporting high-flow gas.

### SUMMARY OF THE INVENTION

An object of the present disclosure is to provide a blood pressure measuring module, which is easily implemented in a blood pressure measurement device. By utilizing an inflatable blood pressure measuring method directly, and combining an optical blood pressure measuring method measured by an optical sensor for calibration, the most accurate information of blood pressure measurement value is obtained. In addition, the information is further transmitted through an external connection device to a self-learning artificial intelligence (AI) program, which is responsible for 24-hour analysis and monitoring. It has advantages of abnormal feedback and notification warnings.

In accordance with an aspect of the present disclosure, a blood pressure measuring module is provided. The blood pressure measuring module includes at least one module body, at least one gas transportation device and at least one sensor. The at least one module body is connected to an airbag to control inflation and deflation operation of the airbag. The at least one gas transportation device controls gas to flow. The at least one sensor measures a gas pressure varied in the airbag or a pressure in contact with a user's skin. The gas transportation device is actuated to transport gas, the gas is introduced into the module body and concentrated in the airbag, and the airbag is inflated for performing a blood pressure measurement, wherein the gas pressure varied in the airbag or the pressure in contact with a user's skin is measured through the sensor, to calculate a blood pressure information of the user under monitoring.

The above contents of the present disclosure will become more readily apparent to those ordinarily skilled in the art after reviewing the following detailed description and accompanying drawings, in which:

### BRIEF DESCRIPTION OF THE DRAWINGS

FIG. 1 is a cross-sectional schematic view illustrating a blood pressure measuring module and a blood pressure measuring device according to an embodiment of the present disclosure;

FIG. 2A is a perspective schematic view illustrating the blood pressure measuring device combined with a wearable component according to an embodiment of the present disclosure;

FIG. 2B is a perspective schematic view illustrating a gas transportation device disposed within the blood pressure measuring device according to an embodiment of the present disclosure;

FIG. 3A is an exploded perspective view illustrating the module body and the gas transportation device of the blood pressure measuring device according to the embodiment of the present disclosure;

FIG. 3B is a top view illustrating a converging plate of the module body in FIG. 3A;

FIG. 3C is a bottom view illustrating a converging plate of the module body in FIG. 3A;

FIG. 3D is a top view illustrating a chamber plate of the module body in FIG. 3A;

FIG. 3E is a bottom view illustrating the chamber plate of the module body in FIG. 3A;

FIG. 3F is a top view illustrating a valve plate of the module body in FIG. 3A;

FIG. 3G is a bottom view illustrating the valve plate of the module body in FIG. 3A;

FIG. 4 is a cross-sectional schematic view illustrating the blood pressure measuring module in FIG. 1 being actuated to inflate the airbag;

FIG. 5A is a cross-sectional schematic view illustrating the airbag of the blood pressure measurement module disposed inside the wearable component according to the embodiment of the present disclosure;

FIG. 5B is a cross-sectional schematic view showing the airbag of the blood pressure measurement module disposed inside the wearable component being actuated to perform inflation;

FIG. 6A is a cross-sectional schematic view illustrating the sensor of the blood pressure measurement module disposed outside the airbag according to the embodiment of the present disclosure;

FIG. 6B is a cross-sectional schematic view showing the sensor of the blood pressure measurement module disposed outside the airbag and the airbag being actuated to perform inflation;

FIG. 6C is a perspective view showing the sensor of the blood pressure measuring module in FIG. 6B performing the blood pressure measurement;

FIG. 7 is a cross-sectional schematic view illustrating the module body assembled with two gas transportation devices;

FIG. 8A is a cross-sectional schematic view illustrating the gas converging action of the blood pressure measuring module in FIG. 7;

FIG. 8B is a cross-sectional schematic view illustrating the gas discharging action of the blood pressure measuring module in FIG. 7;

FIG. 9A is an exploded perspective view illustrating the micro pump of the blood pressure measuring device according to the embodiment of the present disclosure;

FIG. 9B is an exploded perspective view illustrating the micro pump of the blood pressure measuring device from another view angle according to the embodiment of the present disclosure;

FIG. 10A is a cross-sectional view illustrating the micro pump of the blood pressure measuring device according to the embodiment of the present disclosure;

FIG. 10B is a cross-sectional view illustrating the micro pump of the blood pressure measuring device according to another embodiment of the present disclosure;

FIGS. 10C to 10E are cross-sectional views illustrating the actions of the micro pump according to the embodiment of the present disclosure; and

FIG. 11 is a block diagram shows the communicator of the blood pressure measuring module communicated with an external connection device according to the embodiment of the present disclosure.

### DETAILED DESCRIPTION OF THE PREFERRED EMBODIMENT

The present disclosure will now be described more specifically with reference to the following embodiments. It is to be noted that the following descriptions of preferred embodiments of this disclosure are presented herein for purpose of illustration and description only. It is not intended to be exhaustive or to be limited to the precise form disclosed.

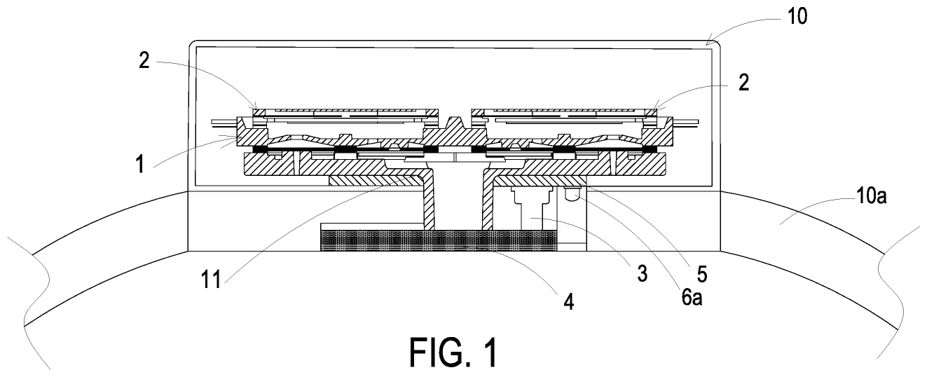

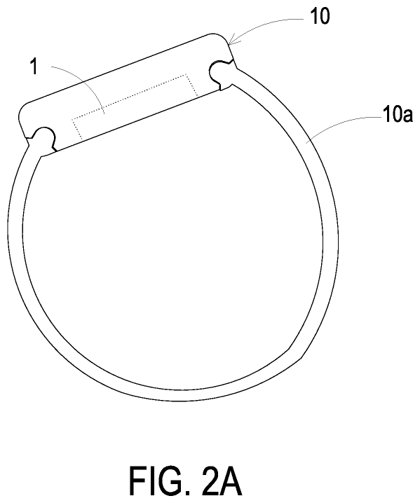

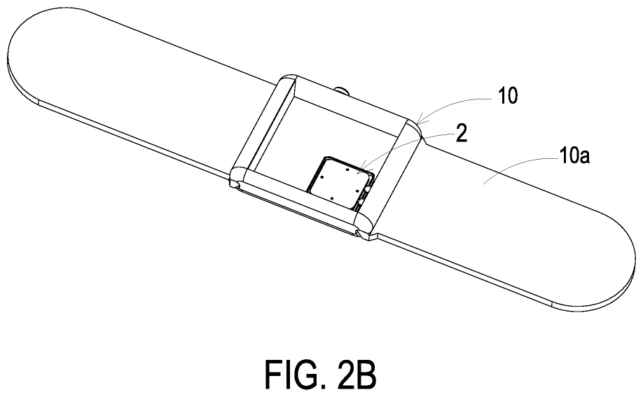

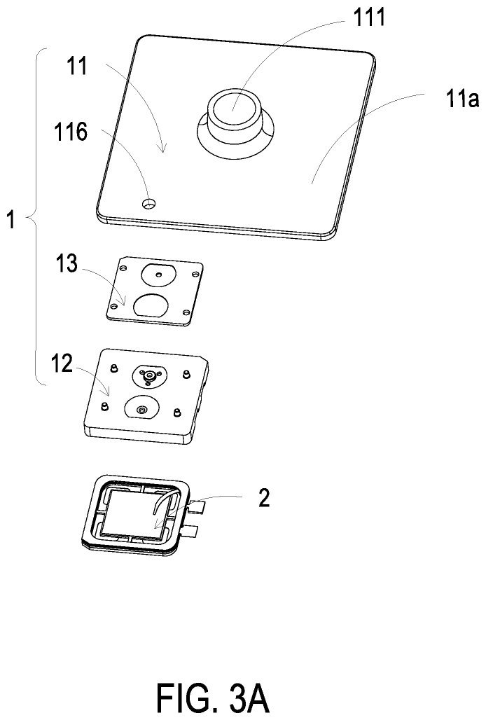

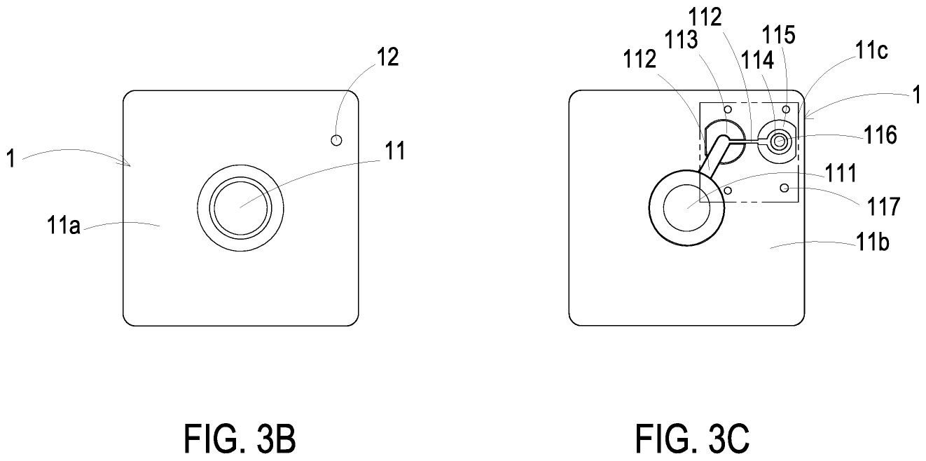

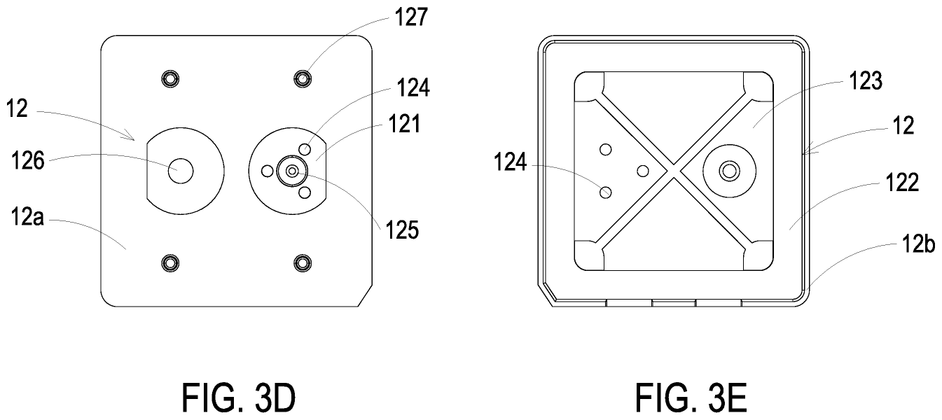

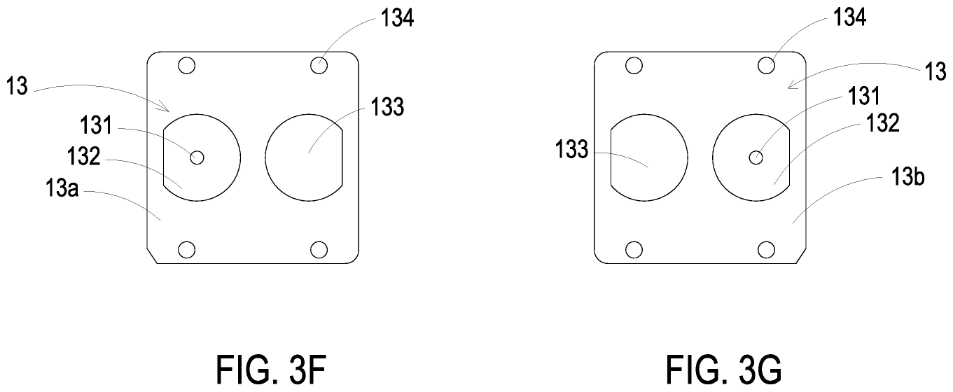

Please refer to FIG. 1, FIG. 2A, FIG. 2B and FIG. 3A. The present discourse provides a blood pressure measuring module including a module body 1, a gas transportation device 2 and a sensor 3. The module body 1 is connected to an airbag 4 to control inflation and deflation operation of the airbag 4. In the embodiment, the module body 1 includes a converging plate 11, at least one chamber plate 12 and at least one valve plate 13. The gas transportation device 2 controls gas to flow. Preferably but not exclusively, the gas transportation device 2 is one selected from the group consisting of a micro pump, an actuator and a quartz oscillator. The sensor 3 measures a gas pressure varied in the airbag 4. In that, the gas transportation device 2 is actuated to transport the gas, the gas is introduced into the module body 1 and concentrated in the airbag 4, and the airbag 4 is inflated for performing a blood pressure measurement. In the embodiment, the gas pressure varied in the airbag 4 or the pressure in contact with a user's skin is measured through the sensor 3, to calculate a blood pressure information of the user under monitoring. Moreover, the gas transportation device 2 is covered on one side of the module body 1, and connected to the airbag 4 with the converging plate 11 of the module body 1 collaboratively to form a blood pressure measuring device 10. Alternatively, as shown in FIGS. 1 and 3A, a converging plate 11, a plurality of chamber plates 12 and a plurality of valve plates 13 of the module body 1 and a plurality of gas transportation devices 2 arranged in parallel are collaboratively connected to an airbag 4 to form a blood pressure measuring device 10. In the embodiment, the numbers of the chamber plate 12, the valve plate 13 and the gas transportation device 2 are all the same, and can be for example but not limited to one. In order to briefly show the structure of the present disclosure, FIG. 3A only representatively shows the structure corresponding to one corner of the converging plate 11. In the embodiment, the blood pressure measuring device 10 is combined with a wearable component 10a, so that the blood pressure measuring device 10 can be wore on the human's body for performing the blood pressure measurement. In the embodiment, the wearable component 10a can be made of a flexible or a hard material, and is configured as an annular-strapped structure. For example, the wearable component 10a can be made of a silicon material, a plastic material, a metal material or other materials, but not limited thereto. The main disposition purpose of the wearable member 102 is to be annularly worn on a specific portion of the user such as wrist, arms and feet, but not limited thereto. Two ends of the wearable component 10a are connected to each other with a Velcro tape, a fastening method which joints a protrusion and a concave, a buckle which is normally used, or even the wearable component 10a can be formed as one piece. The connection method for the two ends of the wearable component 10a can be varied according to the practical requirements, and is not limited thereto.

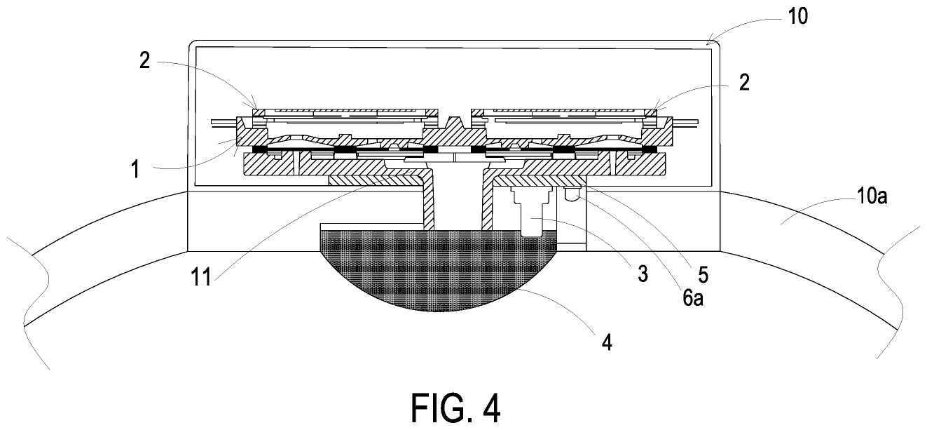

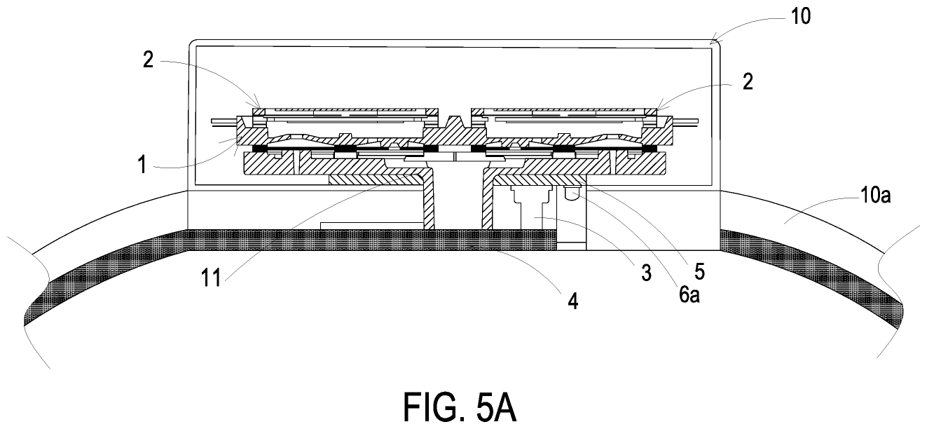

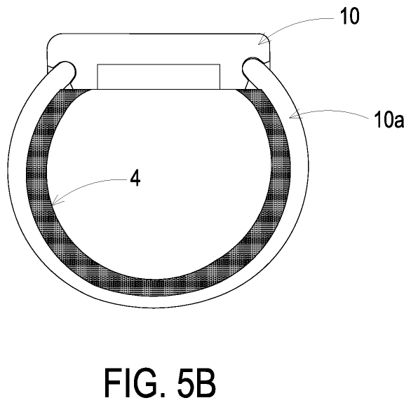

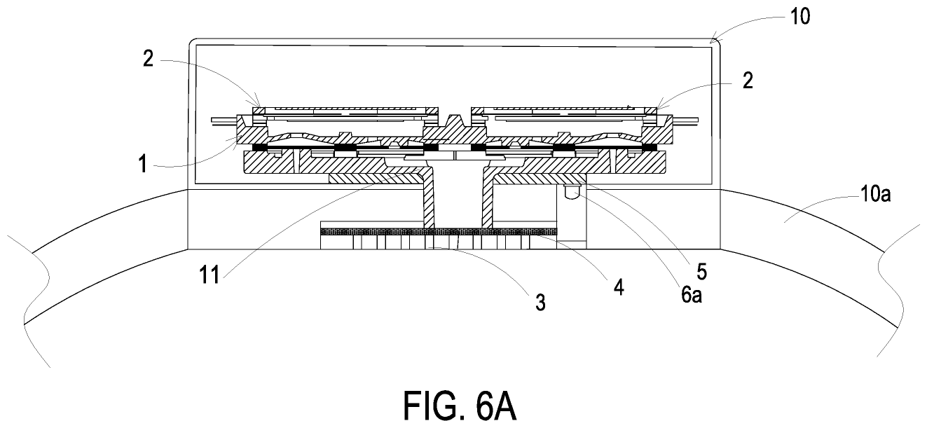

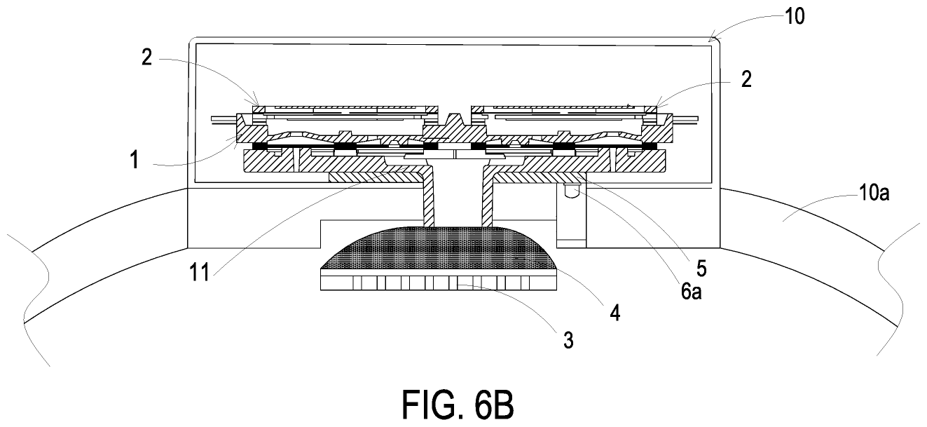

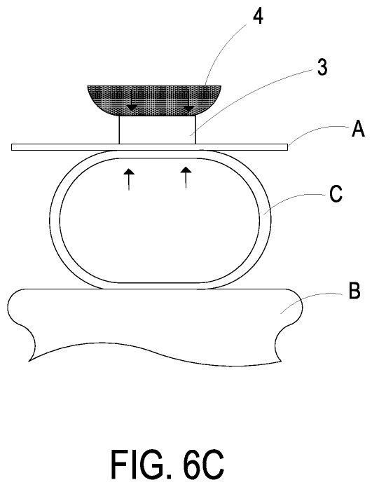

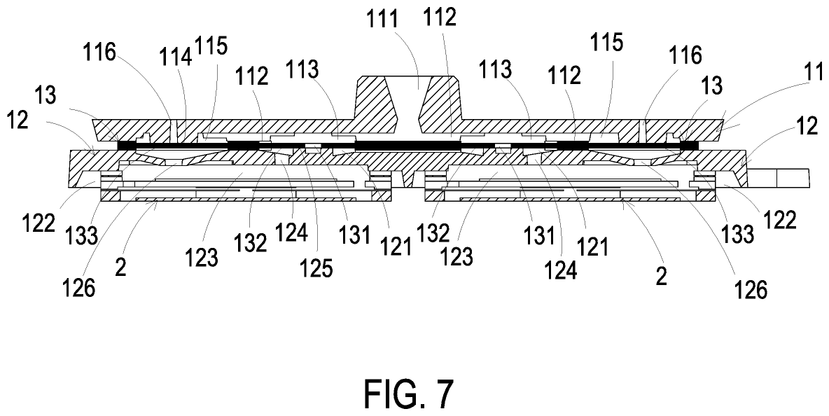

Please refer to FIG. 1. In the embodiment, the airbag 4 is disposed at a bottom of the blood pressure measuring device 10 and contracted and hidden to form a flat surface. When the gas transportation device 2 is driven to transport the gas, the gas is introduced into the module body 1 and accumulated into the airbag 4 to inflate (as shown in FIG. 4) for performing the blood pressure measurement, the gas pressure accumulated in the airbag 4 is measured through the sensor 3, and the blood pressure information of the user under monitoring is calculated. In an embodiment as shown in FIG. 5A, the airbag 4 is disposed within the wearable component 10a, and contracted and hidden to form a flat surface. When the gas transportation device 2 is driven to transport the gas, the gas is introduced into the module body 1 and accumulated into the airbag 4 to inflate (as shown in FIG. 5B) for performing the blood pressure measurement, the gas pressure accumulated in the airbag 4 is measured through the sensor 3, and the blood pressure information of the user under monitoring is calculated. In an embodiment as shown in FIG. 6A, the sensor 3 is disposed on an outside of the airbag 4. Preferably but not exclusively, the sensor 3 is an array-type pressure sensor. When the gas transportation device 2 is driven to transport the gas, the gas is introduced into the module body 1 and accumulated into the airbag 4 to inflate (as shown in FIG. 6B) for performing the blood pressure measurement. The sensor 3 is pressed against the user's skin A and the artery C between the user's bone B and the user' skin A is compressed. As shown in FIG. 6C, while the sensor 3 is pressed against the user's artery C, an applanation scanning is utilized to detect the user's artery C, so as to calculate the blood pressure information of the user under monitoring.

Please refer to FIG. 1 and FIGS. 3A to 3G In the embodiment, the blood pressure measuring module includes a plurality of gas transportation devices 2 arranged in parallel and covered on one side of the module body 1. Preferably but not exclusively, the gas transportation device 2 is a micro pump. The assembly and operation relationship between the gas transportation devices 2 and the module body 1 are described as the following.

In the embodiment, the module body 1 includes a converging plate 11, at least one chamber plate 12 and at least one valve plate 13. The converging plate 11 is connected to the airbag 4, and assembled with and positioned on the chamber plate 12. The valve plate 13 is disposed between the converging plate 11 and the chamber plate 12, so as to control the inflation and deflation operation of the airbag 4.

In the embodiment, the converging plate 11 of the blood pressure measuring module is assembled with the plurality of chamber plates 12 and the plurality of valve plates 13, and further combined with the plurality of gas transportation devices 2. The combinations are collaboratively connected to an airbag 4 to form a blood pressure measuring device 10.

In the embodiment, the converging plate 11 has a converging plate first surface 11a and a converging plate second surface 11b. The converging plate first surface 11a and the converging plate second surface 11b are two surfaces opposite to each other. Moreover, the converging plate 11 is divided a plurality of converging plate mounting sections 11c, which correspond to the plurality of chamber plates 12, the plurality of valve plates 13 and the gas transportation device 2, and is adjustable according to the practical requirements. That is, the converging plate mounting sections 11c with the required number are included on the converging plate 11. In the embodiment, the converging plate 11 has a converging outlet 111 and the converging outlet 111 passes through the converging plate first surface 11a and the converging plate second surface 11b. Each of the converging plate mounting sections 11c includes a converging groove 113, a converging plate protrusion 114, a discharging groove 115 and a discharging outlet 116. The converging groove 113, the converging plate protrusion 114 and the discharging groove 115 are disposed on the converging plate second surface 11b. A guiding groove 112 is disposed on the converging plate second surface 11b and in communication with the converging outlet 111. The guiding groove 112 is served as a communication groove between the converging groove 113 and the discharging groove 115, so that the converging groove 113 and the discharging groove 115 are in communication with each other. In the embodiment, the converging plate protrusion 114 is disposed in and surrounded by the discharging groove 115. The discharging outlet 116 is disposed at a center of the converging plate protrusion 114 and passes through the converging plate first surface 11a and the converging plate second surface 11b. In such a manner, the converging second surface 11b of the converging plate 11 corresponds to and covers the chamber plate 12, the gas outputted from the chamber plate 12 is converged in the guiding groove 112 of the converging plate 11, and the gas converged in the guiding groove 112 is further transported to the converging outlet 111 for output. Notably, when the plurality of converging plate mounting sections 11c with the required number are disposed on the converging plate 11, only one converging outlet 111 is correspondingly disposed on the converging plate 11, and the airbag 4 is collaboratively connected thereto for converging the gas. In addition, there are a plurality of discharging outlets 116 corresponding to the plurality of converging plate mounting sections 11c for pressure relief and discharging the gas.

In the embodiment, each of the chamber plates 12 has a chamber plate first surface 12a and a chamber plate second surface 12b. The chamber plate first surface 12a and the chamber plate second surface 12b are two surfaces opposite to each other. The converging plate 11 is disposed on the chamber plate first surface 12a. A guiding chamber 121 is concavely formed on the chamber plate first surface 12a, and a mounting frame slot 122 is concavely formed on the chamber plate second surface 12b. In the embodiment, the guiding chamber 121 spatially corresponds to and is in communication with the converging groove 113 of the converging plate 11. In other words, the guiding chamber 121 and the mounting frame slot 122 are respectively disposed on two opposite surfaces of the chamber plate 12. In the embodiment, a converging chamber 123 is formed on a bottom of the mounting frame slot 122 and has at least one first communicating hole 124 disposed at the bottom. The first communicating hole 124 runs through the chamber plate first surface 12a and is in communication with the guiding chamber 121. Preferably but not exclusively, in the embodiment, there are three first communicating holes 124. In the embodiment, a chamber plate protrusion 125 is formed in the converging chamber 121 and surrounded by the plurality of first communicating holes 124. Each of the chamber plates 12 further has a second communicating hole 126 corresponding in position to a respective one of the discharging grooves 115 of the converging plate 11, so that the second communicating hole 126 passes through the chamber plate first surface 12a and is in communication with the converging chamber 123.

In the embodiment, the valve plates 13 are disposed between the converging plate 11 and the chamber plates 12. When the valve plates 13 are carried and positioned on the chamber plate first surface 12a of the chamber plate 12, each of the valve plates 13 abuts against a respective one of the chamber plate protrusions 125 of the plurality of chamber plates 12. In the embodiment, each of the valve plates 13 includes a valve hole 131, which is corresponding in position to a respective one of the chamber plate protrusions 125. The valve holes 131 are respectively closed by the chamber plate protrusions 125. On the other hand, the converging plate protrusion 114 of each of the converging plate mounting sections 11c of the converging plate 11 is abutted against by a respective one of the plurality of valve plates 13. In the embodiment, each of the valve plates 13 has a valve plate first surface 13a and a valve plate second surface 13b, and each of the valve plates 13 includes a converging concave portion 132 and a discharging concave portion 133 disposed between the valve plate first surface 13a and the valve plate second surface 13b. Preferably but not exclusively, the converging concave portion 132 and the discharging concave portion 133 do not protrude out of the valve plate first surface 13a and the valve plate second surface 13b, respectively. The converging concave portion 132 abuts against a respective one of the chamber plate protrusions 125 of the plurality of chamber plates 12, so that the valve hole 131 disposed in the converging concave portion 132 is closed by the respective one of the chamber plate protrusions 125. The discharging concave portion 133 abuts against a respective one of the converging plate protrusions 114 of the converging plate mounting sections 11c of the converging plate 11 to close a respective one of the discharging outlets 116.

In order to fixedly position the valve plates 13 between the chamber plates 12 and the converging plate 11, each of the chamber plates 12 further has a plurality of tenons 127 disposed on the chamber plate first surface 12a. The valve plate 13 is disposed on the chamber plate first surface 12a of the chamber plate 12, and each of the valve plates 13 further has a plurality of positioning holes 134 respectively corresponding in position to the plurality of tenons 127. The converging plate 11 is disposed on the valve plate first surfaces 13a of the valve plates 13, and the converging plate 11 further has a plurality of mortises 117 disposed in the converging plate second surface 11b and respectively corresponding in position to the positioning holes 134 of the valve plates 13. When the valve plate 13 is disposed between the converging plate 11 and the chamber plate 12, the tenons 127 of the chamber plate 14 respectively extend through the positioning holes 134 of the valve plate 13 and into the mortises 117 of the converging plate 11 for fixing the valve plate 13.

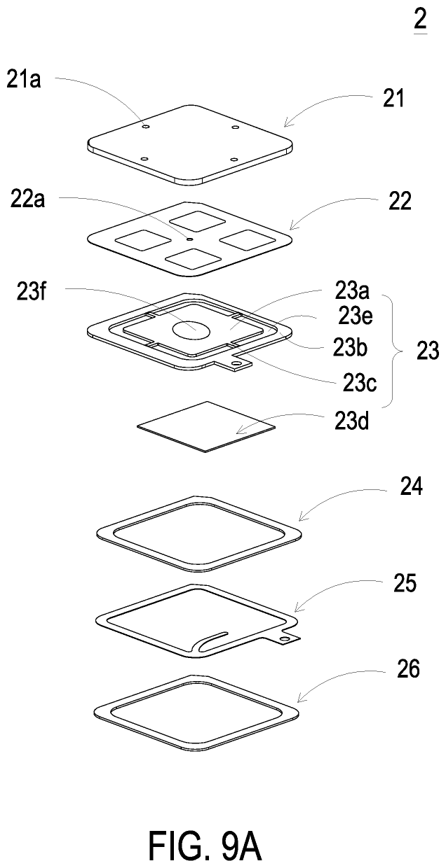

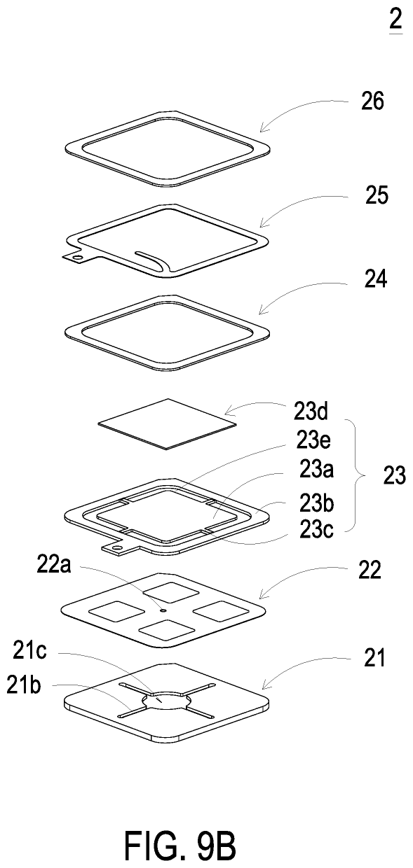

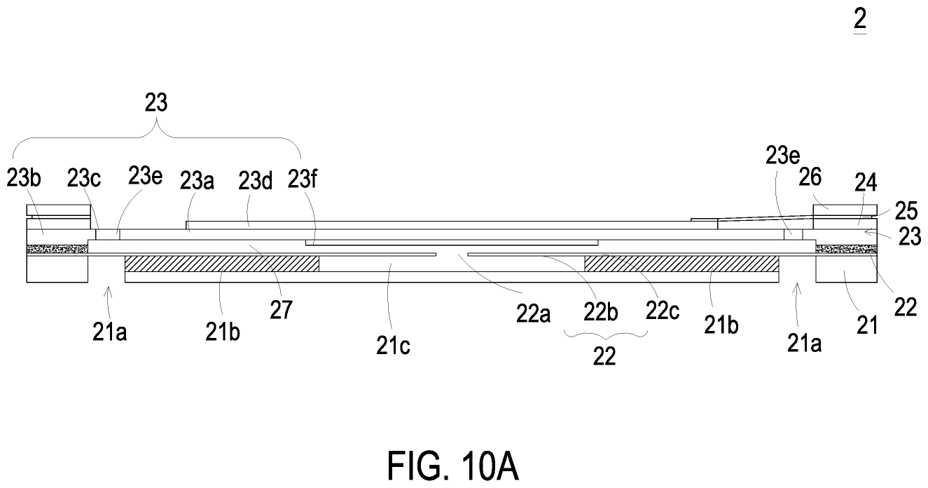

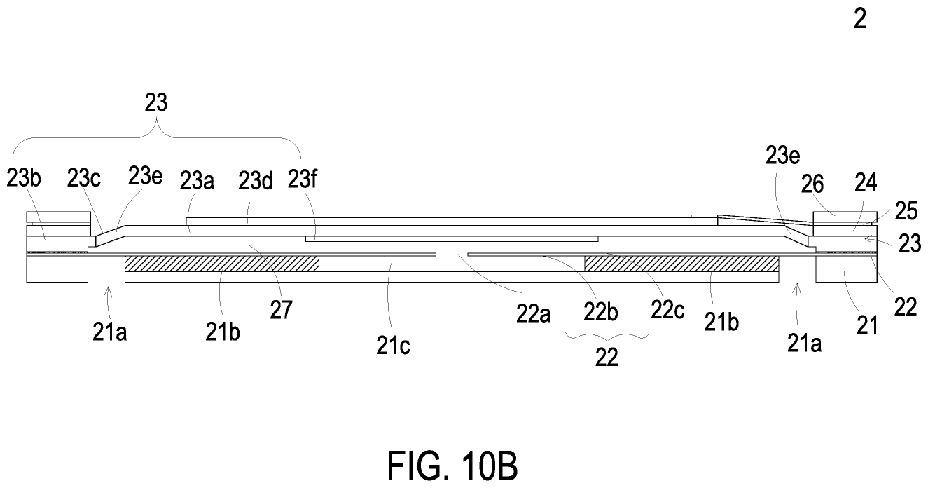

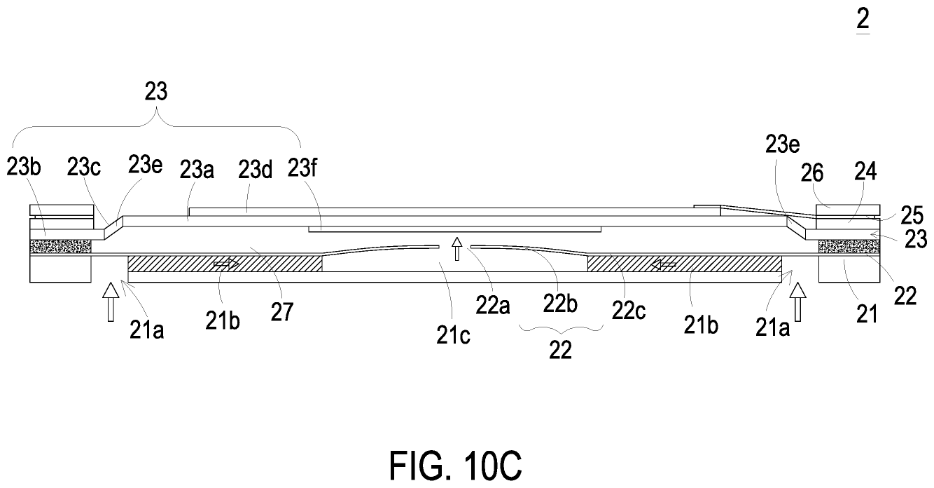

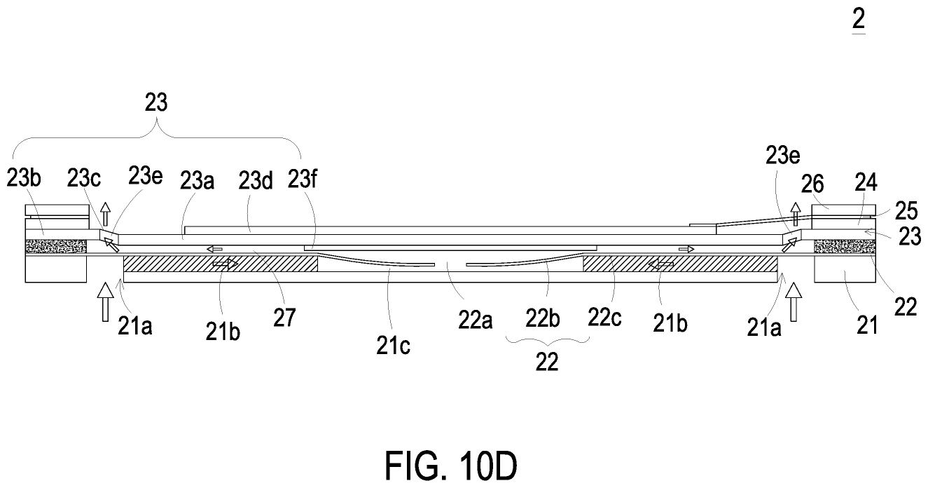

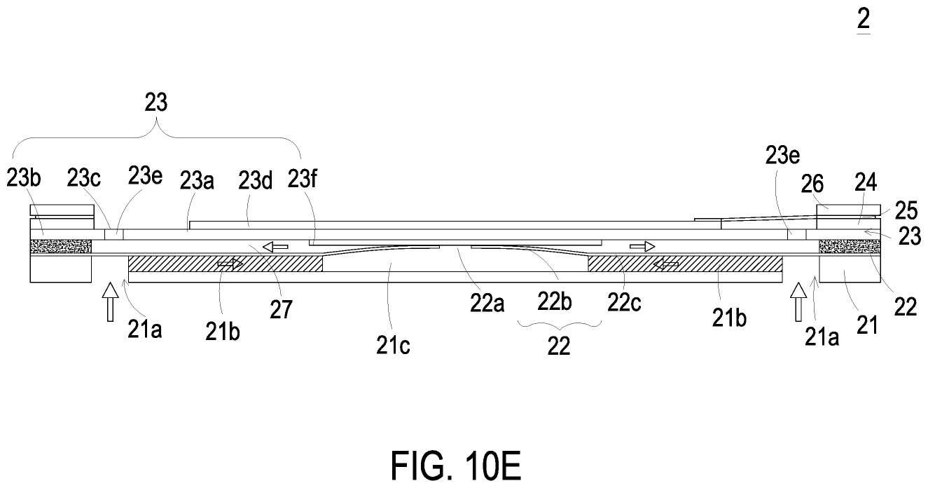

Please refer to FIG. 9A, FIG. 9B and FIGS. 10A to 10E. In the embodiment, the gas transportation device 2 is disposed for outputting the gas, and mounted and positioned in the mounting frame slot 122 of the chamber plate 12 so as to close the converging chamber 123 and transport the gas into the converging chamber 123. In the embodiment, the gas transportation device 2 includes a fluid inlet plate 21, a resonance plate 22, a piezoelectric actuator 23, a first insulating plate 24, a conducting plate 25 and a second insulating plate 26. The fluid inlet plate 21, the resonance plate 22, the piezoelectric actuator 23, the first insulating plate 24, the conducting plate 25 and the second insulating plate 26 are sequentially stacked. The fluid inlet plate 21 has at least one fluid inlet hole 21a, at least one convergence channel 21b, and a convergence chamber 21c. The at least one fluid inlet hole 21a is disposed for introducing the gas. The fluid inlet hole 21a correspondingly passes through the convergence channel 21b, and the convergence channel 21b is converged to the convergence chamber 21c. Thus, the gas introduced through the fluid inlet hole 21a is converged to the convergence chamber 21c. In this embodiment, the number of the at least one fluid inlet hole 21a and the number of the at least one convergence channel 21b are the same, and the fluid inlet plate 21 has four fluid inlet holes 21a and four convergence channels 21b, but not limited thereto. The four fluid inlet holes 21a correspondingly passes through the four convergence channels 21b, and the four convergence channels 21b are converged to the convergence chamber 21c.

In the embodiment, the resonance plate 22 is stickily disposed on the fluid inlet plate 21, and has a central aperture 22a, a movable part 22b and a fixed part 22c. The central aperture 22a is disposed at the center of the resonance plate 22, and is corresponding in position to the convergence chamber 21c of the fluid inlet plate 21. The movable part 22b surrounds the central aperture 22a and corresponds in position to the convergence chamber 21c. The fixed part 22c surrounds the movable part 22b and is fixedly attached on the fluid inlet plate 21.

In the embodiment, the piezoelectric actuator 23 includes a suspension plate 23a, an outer frame 23b, at least one bracket 23c, a piezoelectric element 23d, at least one vacant space 23e and a bulge 23E The suspension plate 23a is square-shaped because the square suspension plate 23a is more power-saving than the circular suspension plate. Generally, the consumed power of the capacitive load at the resonance frequency is positively related to the resonance frequency. Since the resonance frequency of the square suspension plate 23a is obviously lower than that of the circular square suspension plate, the consumed power of the square suspension plate 23a is fewer. Therefore, the square suspension plate 23a in this embodiment has the effectiveness of power-saving. The outer frame 23b surrounds an outer side of the suspension plate 23a. At least one bracket 23c is connected between the suspension plate 23a and the outer frame 23b for providing an elastic support. The piezoelectric element 23d has a side, and a length of the side of the piezoelectric element 23d is less than or equal to that of the suspension plate 23a. The piezoelectric element 23d is attached on a surface of the suspension plate 23a, and when a voltage is applied to the piezoelectric element 23d, the suspension plate 23a is driven to undergo a bending vibration. The at least one vacant space 23e is formed among the suspension plate 23a, the outer frame 23b and the at least one bracket 23c and disposed for allowing the gas to pass through. The bulge 23f is disposed on the other surface of the suspension plate 23a that is opposite to the piezoelectric element 23d. In the embodiment, the bulge 23f is a protruding structure that is formed as one piece on the other surface of the suspension plate 23a opposite to the piezoelectric element 23d, and is formed by an etching process.

In the embodiment, the fluid inlet plate 21, the resonance plate 22, the piezoelectric actuator 23, the first insulating plate 24, the conducting plate 25 and the second insulating plate 26 are sequentially stacked. A chamber space 27 is formed between the suspension plate 23a and the resonance plate 22, and the chamber space 27 can be formed by filling a gap between the resonance plate 22 and the outer frame 23b of the piezoelectric actuator 23 with a material, such as a conductive adhesive, but not limited thereto. Thus, a specific depth between the resonance plate 22 and the suspension plate 23a is maintained to allow the gas to pass rapidly. In addition, since the resonance plate 22 and the suspension plate 23a are maintained at a suitable distance, so that the contact interference therebetween is reduced and the generated noise is largely reduced. In some other embodiments, the thickness of the conductive adhesive filled into the gap between the resonance plate 22 and the outer frame 23b of the piezoelectric actuator 23 is reduced by increasing the height of the outer frame 23b of the piezoelectric actuator 23. In that, the suspension plate 23a and the resonance plate 22 are maintained at a suitable distance and the thickness of conductive adhesive filled in each of the entire gas transportation device 2 is not influenced due to the hot pressing temperature and the cooling temperature. It avoids that the actual size of the chamber space 27 is influenced due to the thermal expansion and contraction after the entire gas transportation device 2 is assembled. In addition, the size of the chamber space 27 will affect the effectiveness of the gas transportation device 2, therefore it is important to maintain the size of the chamber space 27. Please refer to FIG. 9A, in some other embodiments, the suspension plate 23a is formed by stamping to make it extend at a distance in a direction away from the resonance plates 22. The extended distance can be adjusted through the at least one bracket 23c formed between the suspension plate 23a and the outer frame 23b. Consequently, the top surface of the bulge 23f disposed on the suspension plate 23a and a coupling surface of the outer frame 23b are non-coplanar. That is, the top surface of the bulge 23f is away from the resonance plates 22, and the top surface of the bulge 23f and the coupling surface of the outer frame 23b are non-coplanar. By utilizing a small amount of filling materials, such as a conductive adhesive applied to the coupling surface of the outer frame 23b, the piezoelectric actuator 23 is attached to the fixed part 22c of the resonance plate 22 by hot pressing, thereby assembling the piezoelectric actuator 23 and the resonance plates 22 in combination. Thus, the structure of the chamber space 27 is improved by directly stamping the suspension plate 23a of the piezoelectric actuator 23 described above. In this way, the required chamber space 27 can be achieved by adjusting the stamping distance of the suspension plate 22 of the piezoelectric actuator 23. It benefits to simplify the structural design of the chamber space 27, and also achieves the advantages of simplifying the process and shortening the processing time. In addition, the first insulating plate 24, the conducting plate 25 and the second insulating plate 26 are all thin frame-shaped sheets, but are not limited thereto, and are sequentially stacked on the piezoelectric actuator 23 to form the entire structure of micro pump of the gas transportation device 2.

In order to understand the actuations of the gas transportation device 2, please refer to FIGS. 10C to 10E. Please refer to FIG. 10C, when the piezoelectric element 23d of the piezoelectric actuator 23 is deformed in response to an applied voltage, the suspension plate 23a is driven to displace in the direction away from the resonance plate 22. In that, the volume of the chamber space 27 is increased, a negative pressure is formed in the chamber space 27, and the gas in the convergence chamber 21c is introduced into the chamber space 27. At the same time, the resonance plate 22 is in resonance and is thus displaced synchronously. Thereby, the volume of the convergence chamber 21c is increased. Since the gas in the convergence chamber 21c is introduced into the chamber space 27, the convergence chamber 21c is also in a negative pressure state, and the gas is sucked into the convergence chamber 21c through the fluid inlet holes 21a and the convergence channels 21b. Then, as shown in FIG. 10D, the piezoelectric element 23d drives the suspension plate 23a to displace toward the resonance plate 22 to compress the chamber space 27. Similarly, the resonance plate 22 is actuated in resonance to the suspension plate 23a and is displaced. Thus, the gas in the chamber space 27 is further transported to pass through the vacant spaces 23e and it achieves the effectiveness of gas transportation. Finally, as shown in FIG. 10E, when the suspension plate 23a is driven to return to an initial state, the resonance plate 22 is also driven to displace. In that, the resonance plate 22 pushes the gas in the chamber space 27 to the vacant spaces 23e, and the volume of the convergence chamber 21c is increased. Thus, the gas can continuously pass through the fluid inlet holes 21a and the convergence channels 21b, and can be converged in the convergence chamber 21c. By repeating the actuations illustrated in FIGS. 10C to 10E continuously, the gas transportation device 2 can continuously transport the gas at high speed. The gas enters the fluid inlet hole 21a, flows through a flow path formed by the fluid inlet plate 21 and the resonance plate 22 with a pressure gradient, and then is transported upwardly through the vacant spaces 23e. It achieves the gas transporting operation of the gas transportation device 2.

Please refer to FIG. 10A, the fluid inlet plate 21, the resonance plate 22, the piezoelectric actuator 23, the first insulating plate 24, the conducting plate 25 and the second insulating plate 26 of each of the transportation devices 22 can be manufactured by a surface micromachining process of a micro-electromechanical-systems (MEMS). In such a manner, the volume of the gas transportation device 2 can be decreased to form a micro-electromechanical-systems pump.

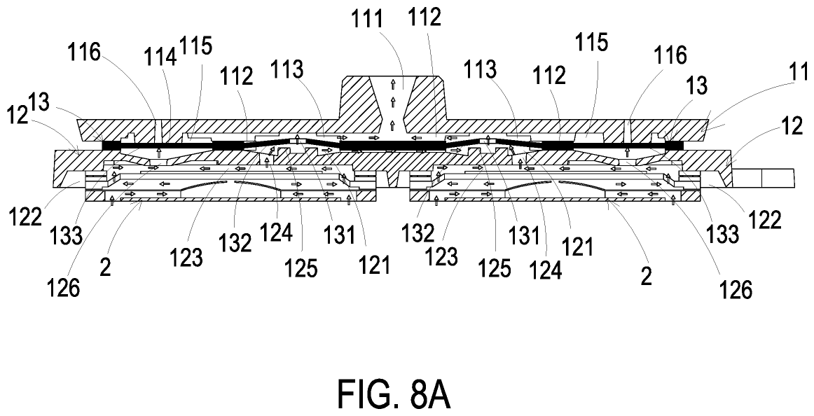

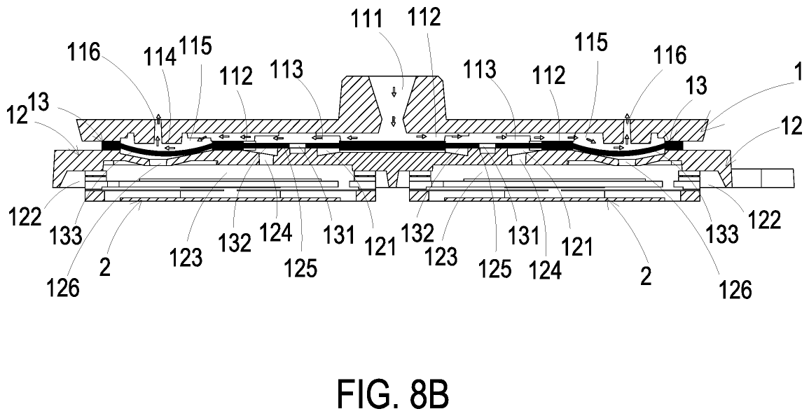

In the embodiment, the plurality of gas transportation devices 2 are arranged in parallel and covered on one side of the module body 1, and connected to the airbag 4 with the converging plate 11, the plurality of chamber plates 12 and the plurality of valve plates 13 of the module body collaboratively to form the blood pressure measuring device 10. As shown in FIG. 8A, when the plurality of gas transportation devices 2 are actuated simultaneously, the gas is inhaled through the converging chamber 123 of each of the chamber plates 12 and flows through the first communicating hole 124 of each of the chamber plates 12. In that, the gas is transported to push each of the valve plates 13 to move away from a corresponding one of the chamber plate protrusions 125. In the embodiment, when the gas pushes the converging concave portion 132 of each of the valve plates 13 to move away from the corresponding one of the chamber plate protrusions 125, the gas passes through the valve holes 131 of the valve plates 13 and is transported to the converging grooves 113 of the converging plate 11. At the same time, the gas in the converging chambers 123 of the chamber plates 12 is transported through the second communicating holes 126 to contact the valve plates 13, so that the discharging concave portions 133 of the valve plates 13 are pushed to abut against the converging plate protrusions 114 of the converging plate 11 respectively so as to close the discharging outlets 116. Then, the gas is converged in the guiding groove 112 through the converging groove 113 and then flows into the converging outlet 111 of the converging plate 11 for discharging. Thus, the gas outputted from the module body 1 is introduced through the converging outlet 111 to the airbag 4, and the airbag 4 is inflated rapidly for performing the blood pressure measurement. Moreover, the sensor 3 measures the gas pressure accumulated in the airbag 4, and the blood pressure information of the user under monitoring is calculated. Please refer to FIG. 8B. When the gas transportation devices 2 are not actuated, the gas in the converging outlet 111 of the converging plate 11 flows into the converging groove 113 through the guiding groove 112 and further flows into the discharging groove 115 through the guiding groove 112. In that, the gas is transported to push the valve plate 13 which corresponds in position to the discharging groove 115 to move away from the corresponding one of the converging plate protrusions 114. In the embodiment, when the gas is transported to push the discharging concave portion 133 of the valve plate 13 to move away from the corresponding one of the converging plate protrusions 114, the discharging outlet 116 is opened. Then, the gas is discharged out of the converging plate 11 through the discharging outlet 116 for pressure relief.

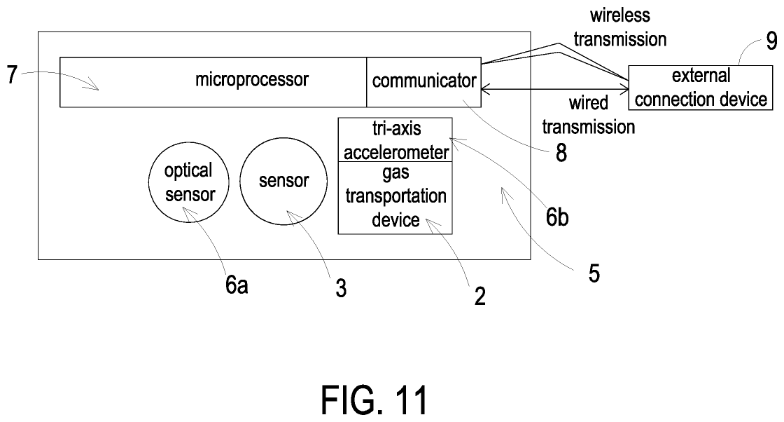

Please refer to FIGS. 1 and 11. In the embodiment, the blood pressure measuring module further includes a driving circuit board 5, an optical sensor 6a, a tri-axial accelerometer 6b, a microprocessor 7 and a communicator 8. The gas transportation device 2, the sensor 3, and the optical sensor 6a, the tri-axial accelerometer 6b, the microprocessor 7 and the communicator 8 are packaged on the driving circuit board 5 for electrical connection. The microprocessor 7 provides driving signals to the gas transportation device 2, the sensor 3, the optical sensor 6a, the tri-axial accelerometer 6b and the communicator 8, controls actuation of the gas transportation device 2, receives measuring signals measured by the sensor 3 and the optical sensor 6a, converts the measuring signals into information data, and transmits the information data through the communicator 8 to an external connection device 9 for storage, recording and carrying out a further analysis, so as to realize a physical health condition of the user much more. In the embodiment, the communicator 8 is configured as a wired transmission module or a wireless transmission module. Preferably but not exclusively, the wired transmission module is one selected from the group consisting of USB, mini-USB and micro-USB. Preferably but not exclusively, the wireless transmission module is one selected from the group consisting of a Wi-Fi module, a Bluetooth module, a radio frequency identification module and a near field communication module. In an embodiment, the communicator 8 further includes a wired transmission module and a wireless transmission module at the same time. The data transmission mode of the communicator 8 is adjustable according to the practical requirements. Any transmission mode for transmitting the physiological information of the user stored in the microprocessor 7 to the external connection device 9 can be implemented in the present disclosure. The present disclosure is not limited thereto, and not redundantly described herein. Preferably but not exclusively, the external connection device 9 is configured as at least one selected from the group consisting of a cloud system, a portable device and a computer system. In the embodiment, the external connection device 9 receives the information data transmitted from the blood pressure measuring module wore by the user, and carries out a further analysis and comparison of the information data through a self-learning artificial intelligence program in a statistical manner. Preferably but not exclusively, the self-learning artificial intelligence program is executed to analyze the information data of the user to generate an upper blood pressure and a lower blood pressure according to a medical standard. When the upper blood pressure and the lower blood pressure are out of range, a warning notice is fed back immediately to the blood pressure measuring device 10, so as to realize the physical health condition of the user much more.

In the embodiment, the optical sensor 6a receives a reflected light from the user's skin tissue irradiated by an emitting light source and generates a detection signal, to achieve a photoplethysmography (PPG) measurement principle. The detection signal is provided to the microprocessor 7 and converted into health data information for output. The health data information includes one selected from the group consisting of heart rate data, electrocardiogram data and blood pressure data. The optical measurement is also a way to achieve blood pressure measurement. Although the optical measurement can be measured every minute and every second at any time, the health data information obtained from the optical measurement is generated through an algorithm adjustment, not directly measured by an inflatable measurement method. The result of the optical measurement is not accurate enough. In view of that, the present disclosure provides the blood pressure measuring module, which is miniaturized and suitable to be worn to achieve an inflatable blood pressure measuring method directly and obtain the most accurate information of blood pressure measurement value. In an embodiment, the accurate blood pressure measuring value is utilized as a calibration basis for an initial measurement of blood pressure of the optical sensor 6a, and heart rate variability (HRV) and atrial fibrillation (AF) are utilized for auxiliary measurement confirmation compensation. That is, when the optical sensor 6a starts the first measurement, the inflatable blood pressure measurement method is implemented firstly in the blood pressure measuring module of the present disclosure, and the obtained health data information is used as the calculation basis for an initial measurement of blood pressure of the optical sensor 6a. The compensation can be executed after each measuring result of the optical sensor 6a, so as to achieve a more accurate measurement of health data information output. In addition, when a specific situation of the user occurs, it can be realized by the blood pressure measuring module. For example, in an embodiment, the tri-axial accelerometer 6b can be utilized for fall detection. A signal detected by the tri-axial accelerometer 6b is directly transmitted to the microprocessor 7 to control the driving of the gas transportation device 2 to inflate the airbag 4 for blood pressure measurement. The sensor 3 measures the gas pressure accumulated in the airbag 4, and the blood pressure information of the user under monitoring is calculated, to realize the blood pressure information of the user. In another embodiment, when the user's abnormal blood pressure or abnormal blood oxygen is sensed by the optical sensor 6a, the microprocessor 7 receives an abnormal condition according to a signal measured by the optical sensor 6a, and directly controls the driving of the gas transportation device 2 to inflate the airbag 4 for blood pressure measuring. The sensor 3 measures the gas pressure accumulated in the airbag 4 or the pressure in contact with the user's skin, and the blood pressure information of the user under monitoring is calculated, to realize the blood pressure information of the user, so that the physical health condition of the user is realized when the specific situation of the user occurs, and warning notifications and treatment measures for first aid can be issued immediately. It is highly industrially utilized.

In the embodiment, when the blood pressure measuring module of the present disclosure is utilized to form the blood pressure measuring device 10 for performing blood pressure measurement, the microprocessor 7 controls the driving of the gas transportation device 2 every five minutes to sixty minutes to inflate the airbag 4 for automatically performing the blood pressure measurement once. Alternatively, the blood pressure measuring device 10 can be set by the user to perform the blood pressure measurement for storage, recording and carrying out the further analysis. In that, a continuous blood pressure monitoring result can be displayed in a convenient way for the user wearing the blood pressure measuring device 10, so as to realize the physical health condition of the user much more. Moreover, the inflatable blood pressure measuring method is utilized directly in the blood pressure measuring module of the present disclosure, and an optical blood pressure measuring method measured by an optical sensor 6a is combined for calibration. The optical blood pressure measuring method is further combined with the self-learning artificial intelligence (AI) program through the external connection device 9. The program can be responsible for 24-hour analysis and monitoring. If there is an abnormal situation, the blood pressure measuring device 10 including the blood pressure measurement module of the present disclosure is fed back to realize, and the gas transportation device 2 is activated to inflate the airbag 4 to perform a precise blood pressure measurement operation. Thus, the accurate blood pressure data information is obtained and provided to the user wearing the blood pressure measuring device 10 for realizing the health condition. If the blood pressure data information is abnormal, warning notifications can be issued immediately. It is highly industrially utilized.

In summary, the present disclosure provides a blood pressure measuring module, which is easily implemented in a blood pressure measurement device. By utilizing an inflatable blood pressure measuring method directly and combining an optical blood pressure measuring method measured by an optical sensor for calibration, the most accurate information of blood pressure measurement value is obtained. In addition, the information is further transmitted through an external connection device to a self-learning artificial intelligence (AI) program, which is responsible for 24-hour analysis and monitoring. It has functions of abnormal feedback and notification warnings, and is highly industrially utilized.

While the disclosure has been described in terms of what is presently considered to be the most practical and preferred embodiments, it is to be understood that the disclosure needs not be limited to the disclosed embodiments. On the contrary, it is intended to cover various modifications and similar arrangements included within the spirit and scope of the appended claims which are to be accorded with the broadest interpretation so as to encompass all such modifications and similar structures.

What is claimed is:

1. A blood pressure measuring module comprising:
at least one module body connected to an airbag to control inflation and deflation operation of the airbag;
at least one gas transportation device for controlling gas to flow; and
at least one sensor for measuring a gas pressure varied in the airbag or a pressure in contact with a user's skin;
wherein the gas transportation device is actuated to transport the gas, the gas is introduced into the module body and concentrated in the airbag, and the airbag is expanded for performing a blood pressure measurement, wherein the gas pressure varied in the airbag or the pressure in contact with the user's skin is measured through the sensor, to calculate a blood pressure information of the user under monitoring.

2. The blood pressure measuring module according to claim 1, wherein the gas transportation device is one selected from the group consisting of a micro pump, an actuator and a quartz oscillator, wherein the blood pressure measuring module comprises a plurality of gas transportation devices, and the gas transportation devices are arranged in parallel and covered on one side of the module body, and are connected to the airbag with the module body collaboratively to form a blood pressure measuring device.

3. The blood pressure measuring module according to claim 2, wherein the module body comprises:
a converging plate having a converging outlet and a guiding groove in communication with each other, wherein the converging plate is divided into a plurality of converging plate mounting sections, each of the plurality of converging plate mounting sections comprises a converging groove, a converging plate protrusion, a discharging groove and a discharging outlet, the guiding groove is in communication with the converging groove of each of the plurality of converging plate mounting sections, and the converging groove and the discharging groove are in communication with each other, wherein the converging plate protrusion is disposed in and surrounded by the discharging groove, and the discharging outlet is disposed at a center of the converging plate protrusion; and
a plurality of chamber plates, wherein the converging plate is disposed on each of the plurality of chamber plates, the plurality of chamber plates are respectively corresponding in position to the converging plate mounting sections of the converging plate, and a guiding chamber and a mounting frame slot are concavely formed on each of the plurality of chamber plates, wherein the guiding chamber spatially corresponds to and is in communication with the converging groove of the converging plate, the guiding chamber and the mounting frame slot are respectively disposed on two opposite surfaces of the chamber plate, a converging chamber is formed on a bottom of the mounting frame slot and has at least one first communicating hole running through the chamber plate and in communication with the guiding chamber, a chamber plate protrusion is formed in the converging chamber and surrounded by the at least one first communicating hole, and each of the plurality of chamber plates further has a second communicating hole corresponding in position to a respective one of the discharging grooves of the converging plate, wherein the second communicating hole is in communication with the converging chamber; and
a plurality of valve plates, wherein each of the plurality of valve plates is disposed between the converging plate and a respective one of the plurality of chamber plates, and abuts against a respective one of the chamber plate protrusions of the plurality of chamber plates, wherein each of the valve plates comprises a valve hole, which is corresponding in position to the respective one of the chamber plate protrusions of the plurality of chamber plates, the valve holes are respectively closed by the chamber plate protrusions, and the converging plate protrusion of each of the converging plate mounting sections of the converging plate is abutted against by a respective one of the plurality of valve plates so that the discharging outlet is closed by the respective one of the plurality of valve plates;
wherein the plurality of gas transportation devices are disposed and fixed in the mounting frame slot of a respective one of the plurality of chamber plates so as to close the converging chamber and transport the gas into the converge chamber, wherein when the plurality of gas transportation devices are actuated simultaneously, the gas is inhaled through the converging chamber of each of the plurality of the chamber plates, the gas is transported to push each of the plurality of valve plates to move away from a corresponding one of the chamber plate protrusions, the gas passes through the valve holes of the plurality of valve plates and is transported to the converging grooves of the converging plate in communication with the guiding chamber, and the gas is converged in the guiding groove to flow into the converging outlet for discharging.

4. The blood pressure measuring module according to claim 3, wherein when the plurality of gas transportation devices are not actuated, the gas in the converging outlet of the converging plate flows into the converging groove through the guiding groove, the gas is transported to push the valve plate which corresponds in position to the discharging groove to move away from the corresponding one of the converging plate protrusions, so that the discharging outlet is opened, wherein the gas is discharged out of the converging plate from the discharging outlet for pressure relief, wherein each of the plurality of valve plates has a valve plate first surface and a valve plate second surface, and each of the plurality of valve plates is corresponding in position to a respective one of the plurality of chamber plates and comprises a converging concave portion and a discharging concave portion disposed between the valve plate first surface and the valve plate second surface, the converging concave portion and the discharging concave portion do not protrude out of the valve plate first surface and the valve plate second surface respectively, the converging concave portion abuts against a respective one of the chamber plate protrusions of the plurality of chamber plates so that the valve hole disposed in the converging concave portion is closed by the respective one of the chamber plate protrusions, and the discharging concave portion abuts against a respective one of the converging plate protrusions of the converging plate to close a respective one of the discharging outlets.

5. The blood pressure measuring module according to claim 2, wherein each of the plurality of gas transportation devices is a micro pump comprising:
a fluid inlet plate having at least one fluid inlet hole, at least one convergence channel, and a convergence chamber, wherein the at least one fluid inlet hole is disposed to inhale the gas, the at least one convergence channel is in communication with the convergence chamber, and the at least one convergence channel is disposed corresponding in position to the fluid inlet hole to guide the gas from the fluid inlet hole to the convergence chamber;
a resonance plate disposed on the fluid inlet plate and having a central aperture, a movable part and a fixed part, wherein the central aperture is disposed at a center of the resonance plate, and corresponds in position to the convergence chamber of the fluid inlet plate, the movable part surrounds the central aperture and corresponds in position to the convergence chamber, and the fixed part surrounds the movable part and is fixedly attached on the fluid inlet plate; and
a piezoelectric actuator disposed on the resonance plate;
wherein, a chamber space is formed between the resonance plate and the piezoelectric actuator, so that when the piezoelectric actuator is driven, the gas introduced from the at least one fluid inlet hole of the fluid inlet plate is converged to the convergence chamber through the at least one convergence channel, and flows through the central aperture of the resonance plate so as to produce a resonance by the movable part of the resonance plate and the piezoelectric actuator to transport the gas.

6. The blood pressure measuring module according to claim 5, wherein the piezoelectric actuator comprises:
a suspension plate being square-shaped and being permitted to undergo a bending vibration;
an outer frame surrounding the suspension plate;
at least one bracket connected between the suspension plate and the outer frame for providing an elastic support; and
a piezoelectric element having a side, wherein a length of the side of the piezoelectric element is less than or equal to that of the suspension plate, and the piezoelectric element is attached on a surface of the suspension plate, wherein when a voltage is applied to the piezoelectric element, the suspension plate is driven to undergo the bending vibration.

7. The blood pressure measuring module according to claim 6, wherein the suspension plate comprises a bulge, which is integrally formed by an etching process, and disposed on the other surface of the suspension plate that is opposite to the piezoelectric element.

8. The blood pressure measuring module according to claim 2, wherein the gas transportation device is a micro-electromechanical-systems (MEMS) micro pump.

9. The blood pressure measuring module according to claim 2, wherein the airbag is disposed at a bottom of the blood pressure measuring device and contracted and hidden to form a flat surface, and the sensor is disposed on an outside of the airbag, wherein the sensor is an array-type pressure sensor, and an applanation scanning is utilized to detect the user's artery, so as to calculate the blood pressure information of the user under monitoring.

10. The blood pressure measuring module according to claim 2, wherein the blood pressure measuring device is combined with a wearable component, and the airbag is disposed within the wearable component, and contracted and hidden to form a flat surface, wherein the wearable component is a flexible ring-shaped belt structure, so as to be wrapped around one selected from the group consisting of the user's wrist, arms and feet.

11. The blood pressure measuring module according to claim 2, wherein the blood pressure measuring device is combined with a wearable component, and the airbag is disposed within the wearable component, and contracted and hidden to form a flat surface, wherein the wearable component is a ring-shaped belt structure made by a hard material, so as to be wrapped around one selected from the group consisting of the user's wrist, arms and feet.

12. The blood pressure measuring module according to claim 2, wherein the blood pressure measuring module further comprises a driving circuit board, an optical sensor, a tri-axial accelerometer, a microprocessor and a communicator, wherein the gas transportation device, the sensor, the optical sensor, the tri-axial accelerometer, the microprocessor and the communicator are packaged on the driving circuit board for electrical connection, wherein the microprocessor provides driving signals to the plurality of gas transportation devices, the sensor, the optical sensor, the tri-axial accelerometer and the communicator, controls actuation of the plurality of gas transportation devices, receives measuring signals measured by the sensor and the optical sensor, converts the measuring signals into information data, and transmits the information data through the communicator to an external connection device for storage, recording and carrying out a further analysis, so as to realize a physical health condition of the user.

13. The blood pressure measuring module according to claim 12, wherein the communicator is configured as a wired transmission module or a wireless transmission module, wherein the wired transmission module is one selected from the group consisting of USB, mini-USB and micro-USB, wherein the wireless transmission module is one selected from the group consisting of a Wi-Fi module, a Bluetooth module, a radio frequency identification module and a near field communication module, and wherein the external connection device is configured as at least one selected from the group consisting of a cloud system, a portable device and a computer system.

14. The blood pressure measuring module according to claim 12, wherein the external connection device receives the information data transmitted from the blood pressure measuring module wore by the user, and carries out a further analysis and comparison of the information data through a self-learning artificial intelligence program in a statistical manner.

15. The blood pressure measuring module according to claim 14, wherein the self-learning artificial intelligence program is executed to analyze the information data of the user to generate an upper blood pressure and a lower blood pressure according to a medical standard, wherein when the upper blood pressure and the lower blood pressure are out of range, a warning notice is fed back immediately to the blood pressure measuring device, so as to realize the physical health condition of the user.

16. The blood pressure measuring module according to claim 12, wherein the optical sensor receives a reflected light from the user's skin tissue irradiated by an emitting light source and generates a detection signal, which is provided to the microprocessor and converted into health data information for output, wherein the health data information includes one selected from the group consisting of heart rate data, electrocardiogram data and blood pressure data.

17. The blood pressure measuring module according to claim 16, wherein the blood pressure measuring device drives the gas transportation device to inflate the airbag, and the gas pressure accumulated in the airbag is measured through the sensor to calculate an accurate blood pressure measuring value of the user under monitoring, wherein the accurate blood pressure measuring value is utilized as a calibration basis for an initial measurement of blood pressure of the optical sensor, and heart rate variability (HRV) and atrial fibrillation (AF) are utilized for auxiliary measurement confirmation compensation, so as to achieve measurement of the health data information for output.

18. The blood pressure measuring module according to claim 12, wherein a signal detected by the tri-axial accelerometer is directly transmitted to the microprocessor to control the driving of the gas transportation device to inflate the airbag for blood pressure measurement, wherein the sensor measures the gas pressure accumulated in the airbag, and the blood pressure information of the user under monitoring is calculated, to realize the blood pressure information of the user, so that the physical health condition of the user is realized when a specific situation occurs, and notification warnings and treatment measures for first aid are issued immediately.

19. The blood pressure measuring module according to claim 12, wherein when the user's abnormal blood pressure or abnormal blood oxygen is sensed by the optical sensor, the microprocessor receives an abnormal condition according to a signal measured by the optical sensor, and directly controls the driving of the gas transportation device to inflate the airbag for blood pressure measuring, wherein the sensor measures the gas pressure accumulated in the airbag, and the blood pressure information of the user under monitoring is calculated, to realize the blood pressure information of the user, so that the physical health condition of the user is realized when a specific situation occurs, and notification warnings and treatment measures for first aid are issued immediately.

20. The blood pressure measuring module according to claim 12, wherein the microprocessor controls the driving of the gas transportation device every five minutes to sixty minutes to inflate the airbag for automatically performing the blood pressure measurement once, wherein the gas accumulated in the airbag is measured through the sensor to obtain the measuring signal, the microprocessor receives the measuring signal measured by the sensor and converts the measuring signal into the information data, the information data is transmitted to the external connection device through the communicator for storage, recording and carrying out the further analysis, and a continuous blood pressure result is displayed for the user wearing the blood pressure measuring device, so as to realize the physical health condition of the user.
